(19) United States
(12) Patent Application Publication
(10) Pub. No.: US 2024/0068036 A1
(43) Pub. Date: Feb. 29, 2024

(54) BIOMARKER FOR PREDICTING AGE IN DAYS OF PIGS, AND PREDICTION METHOD

(71) Applicant: AGRICULTURAL GENOMICS INSTITUTE AT SHENZHEN, CHINESE ACADEMY OF AGRICULTURAL SCIENCES, Shenzhen (CN)

(72) Inventors: Zhonglin TANG, Shenzhen (CN); Yalan YANG, Shenzhen (CN); Xinhao FAN, Shenzhen (CN); Muya CHEN, Shenzhen (CN)

(21) Appl. No.: 18/502,378

(22) Filed: Nov. 6, 2023

Related U.S. Application Data
(63) Continuation of application No. 18/053,035, filed on Nov. 7, 2022.
Application No. 18/053,035 is a continuation of application No. PCT/CN2020/110263, filed on Aug. 20, 2020.

(30) Foreign Application Priority Data
Jul. 31, 2020 (CN) 202010760582.9

(51) Int. Cl. C12Q 1/6883

(52) U.S. Cl. CPC C12Q 1/6883; C12Q 1/6869

(57) ABSTRACT

Disclosed are biomarkers and a prediction method for predicting age in days in pigs. The biomarkers for predicting age in days of pigs include one or more CpG sites with different methylation levels, and the different methylation levels of the CpG sites correspond to different ages in days of pigs. An Elastic Net linear regression model is constructed by using the methylation levels of the CpG sites and the weights corresponding to each CpG site, thereby predicting age in days of pigs to be tested. The above prediction method has high accuracy, and is accurate and reliable in detecting age in days of pigs, which fills the gap in the age prediction model of pigs based on DNA methylation, and provides an ideal model for investigating important scientific issues such as development and aging of human and animals.

### CROSS-REFERENCE TO RELATED APPLICATIONS

This application is a continuation of U.S. application Ser. No. 18/053,035, filed on Nov. 7, 2022, which is a continuation of International Application No. PCT/CN2020/110263, filed on Aug. 20, 2020, which claims priority to Chinese Patent Application No. 202010760582.9, filed on Jul. 31, 2020. The disclosures of the aforementioned applications are hereby incorporated by reference in their entireties.

### TECHNICAL FIELD

The present application relates to the technical field of detecting age in days of animals, in particular to biomarkers for predicting age in days of pigs, a reagent and a kit for predicting age in days of pigs, and a method for predicting age in days of pigs.

### BACKGROUND ART

The development of precise markers for estimating biological age of humans and animals and assessing the impact of different interventions on lifespan has been a hotspot in development and aging research field. In previous studies, various biomarkers are used to predict age, including telomere length, mutation accumulation, level of gene expression, or T cell specific DNA rearrangement. However, these methods are relatively limited in their ability and accuracy in assessing the aging process due to large differences in the detected ages. Studies on DNA methylation provide new ideas for accurately estimating the age of an organism. In mammals, methylation of numerous CpG sites has been found to be highly correlated with age. These association sites can be used to construct models (called epigenetic clocks), and may be used as biomarkers to quantitatively predict age, thereby addressing some of the key scientific questions in development, aging research, and related fields.

DNA methylation is by far the most accurate biomarker known to predict age. Methylation sites in human saliva samples are firstly used by researchers for age prediction, and methylation markers based on different tissues and blood are developed later. Recently, age prediction models based on DNA methylation levels of a small number of CpG sites have been successively established in mice, wolves, dogs, whales and other species, but age prediction models based on DNA methylation levels in pigs have not yet been reported.

### SUMMARY

In a first aspect of the present application, provided are biomarkers for predicting age in days of pigs, which includes one or more CpG sites with different methylation levels, and the different methylation levels of the CpG sites correspond to different ages of pigs.

Further, on the basis of the technical solution provided in this application, the position information of the CpG site(s) includes any one, preferably more, most preferably 75 of the followings: chr1:265469121, chr1:6993958, chr1:77278255, chr1:77278255, chr1:90279146, chr1:10222822, chr1:200765194, chr1:252703561, chr1:127811329, chr1:218682018, chr1:272166208, chr2:112726051, chr2:131821312, chr3:79519033, chr3:71354421, chr3:96708114, chr3:4786944, chr4:110707399, chr4:51236025, chr4:61693637, chr4:35277986, chr4:71941843, chr4:38392750, chr5:46167692, chr5:3442060, chr5:83823568, chr5:86678792, chr6:63915584, chr6:98241827, chr6:7667231, chr6:59654560, chr6:148902979, chr6:131779338, chr6:131779339, chr6:63915581, chr6:151183086, chr6:107410789, chr6:134649996, chr7:15916877, chr7:1722548, chr7:89164845, chr7:14846023, chr7:70113867, chr7:89164756, chr7:86102364, chr7:89164755, chr8:46226086, chr8:71696260, chr8:138571452, chr8:78759323, chr8:116621205, chr8:41380820, chr9:116669694, chr9:68467395, chr9:96069192, chr9:36094595, chr9:73739560, chr9:114311129, chr10:14130890, chr10:14130912, chr10:27158773, chr11:43923343, chr11:13802486, chr12:52792396, chr13:158289588, chr13:32034512, chr13:77838609, chr13:30455076, chr13:85584193, chr13:1535436, chr13:111038503, chr14:31839031, chr14:71122259, chr16:57712066, chr17:43961681, chr18:17893916.

Further, the biomarkers also include the weights of the CpG sites.

Skeletal muscle accounts for 45%-60% of the body weight of an animal, and consists of skeletal muscle fibers. It is the most abundant tissue in animals and one of the most important production traits for the growth and development of livestock and poultry. The level of meat production performance and meat quality of pig animals depends on the growth and development of individual skeletal muscles of animals.

Muscle development in pigs is a very complex process, including the proliferation of the number of muscle fibers before birth, the increase in the volume of muscle fibers and the transformation of muscle fiber types after birth. This process is regulated by the expression of many genes and transcription factors, and DNA methylation and post-transcriptional regulatory modifications also play an important role. An in-depth understanding of the developmental mechanism of pig skeletal muscle is of great significance for improving the breeding efficiency of pig meat production traits and cultivating high-yield and high-quality new breeds (lines) of pigs. It has important strategic significance and market prospects for ensuring China's food security, realizing the sustainable development of the pig breeding industry, and enhancing international competitiveness.

With respect to the above-mentioned biomarkers provided in this application, the methylation of CpG sites is closely related to the growth and development of mammals, and may be used to predict the growth age in days of pigs, which provides a new idea for the study of the mechanism of meat production traits in pigs, thereby facilitating the molecular design breeding for pigs.

In a second aspect of the present application, provided is a reagent or a kit for predicting age in days of pigs, including a reagent capable of detecting the biomarkers mentioned above, and optional instructions.

In addition, the reagent and kit may also optionally include a reagent for detecting age in days of pigs. For example, a reagent for extracting pig genomic DNA, a reagent for gene sequencing, a reagent for detecting gene methylation levels, and other reagents, consumables or instructions that can be thought of by those skilled in the art.

In a third aspect of the present application, provided is a method for predicting age in days of pigs, including measuring the methylation levels of the biomarker CpG sites in genomic DNA of the pig, and optionally further including utilizing a statistical prediction algorithm to determine the age in days of the pig. Exemplarily, said algorithm includes: (a) obtaining a linear combination of methylation levels of the biomarker CpG sites, and (b) applying a transformation to the linear combination to determine the age in days of the pig.

Further, on the basis of the technical solutions provided in this application, wherein the biomarker CpG site(s) is one or more of the above mentioned 75 biomarker CpG sites.

Further, the biomarker CpG sites include but are not limited to: at least 10, or at least 20, or at least 30, or at least 40, or at least 50, or at least 60, or at least 70, or at least 75 methylation biomarkers.

Further, on the basis of the technical solutions provided in this application, the methylation levels of the CpG sites and the corresponding weights of each CpG site are used to construct an Elastic Net linear regression model for predicting age in days of pigs to be tested.

Further, the required CpG sites for the model are the above 75 CpG sites, and/or the version of pigs reference genome in use is Sscrofa11.1 version.

In the present application, by utilizing the DNA methylation data of the whole genome of pig muscles at different development stages, provided is a method for accurately predicting the growth age in days of pigs based on the methylation levels of one or more of the 75 CpG sites, preferably the 75 CpG sites.

The above-mentioned method for predicting the growth age in days of pigs based on CpG methylation not only provides a new idea for the study of the mechanism of pig meat production traits, but also is beneficial to the molecular design breeding of pigs. Since pigs are closely related to humans, this method provides an ideal model for studying important scientific issues such as development and aging of humans and animals.

Further, on the basis of the technical solutions provided by this application, the CpG sites and the corresponding weight information are shown in the table below:

| Number (i) | CpG position information (β) | Weight (w) |
|---|---|---|
| 1 | chr1: 265469121 | −0.19791914 |
| 2 | chr1: 6993958 | −3.224485644 |
| 3 | chr1: 77278255 | −13.28624592 |
| 4 | chr1: 90279146 | −9.413975275 |
| 5 | chr1: 10222822 | −2.319516222 |
| 6 | chr1: 200765194 | 6.224564956 |
| 7 | chr1: 252703561 | −10.29425473 |
| 8 | chr1: 127811329 | −0.288286911 |
| 9 | chr1: 218682018 | −8.74861671 |
| 10 | chr1: 272166208 | −0.958636654 |
| 11 | chr2: 112726051 | −0.00030695 |
| 12 | chr2: 131821312 | −1.487907119 |
| 13 | chr3: 79519033 | −1.427572944 |
| 14 | chr3: 71354421 | −14.56809668 |
| 15 | chr3: 96708114 | −5.697719601 |
| 16 | chr3: 4786944 | −6.781267851 |
| 17 | chr4: 110707399 | −0.007481015 |
| 18 | chr4: 51236025 | −1.595911641 |
| 19 | chr4: 61693637 | −1.027410147 |
| 20 | chr4: 35277986 | −0.049404384 |
| 21 | chr4: 71941843 | −13.62773853 |
| 22 | chr4: 38392750 | −0.043794313 |
| 23 | chr5: 46167692 | −2.61890723 |
| 24 | chr5: 3442060 | −14.13370338 |
| 25 | chr5: 83823568 | −1.940844913 |
| 26 | chr5: 86678792 | −8.038210429 |
| 27 | chr6: 63915584 | −6.430323147 |
| 28 | chr6: 98241827 | −19.83015838 |
| 29 | chr6: 7667231 | −0.115183771 |
| 30 | chr6: 59654560 | −0.010556261 |
| 31 | chr6: 148902979 | −13.09889713 |
| 32 | chr6: 131779338 | −0.016545453 |
| 33 | chr6: 131779339 | −2.563888441 |
| 34 | chr6: 63915581 | −7.790688318 |
| 35 | chr6: 151183086 | −2.317710899 |
| 36 | chr6: 107410789 | −7.746859508 |
| 37 | chr6: 134649996 | −42.41052359 |
| 38 | chr7: 15916877 | −5.765286814 |
| 39 | chr7: 1722548 | −1.232989258 |
| 40 | chr7: 89164845 | −1.78588923 |
| 41 | chr7: 14846023 | −1.915909405 |
| 42 | chr7: 70113867 | −5.225256985 |
| 43 | chr7: 89164756 | −0.102078131 |
| 44 | chr7: 86102364 | −1.624811107 |
| 45 | chr7: 89164755 | −4.012719139 |
| 46 | chr8: 46226086 | −3.368393933 |
| 47 | chr8: 71696260 | −17.09415973 |
| 48 | chr8: 138571452 | −19.74938423 |
| 49 | chr8: 78759323 | −5.382316805 |
| 50 | chr8: 116621205 | −4.395514047 |
| 51 | chr8: 41380820 | −0.033290161 |
| 52 | chr9: 116669694 | −0.979621002 |
| 53 | chr9: 68467395 | −1.528021515 |
| 54 | chr9: 96069192 | −9.073121614 |
| 55 | chr9: 36094595 | −15.79167462 |
| 56 | chr9: 73739560 | −1.061762087 |
| 57 | chr9: 114311129 | −0.276923385 |
| 58 | chr10: 14130890 | −0.047930706 |
| 59 | chr10: 14130912 | −0.872727299 |
| 60 | chr10: 27158773 | −8.310078727 |
| 61 | chr11: 43923343 | −5.381489916 |
| 62 | chr11: 13802486 | −2.727387937 |
| 63 | chr12: 52792396 | −6.930884723 |
| 64 | chr13: 158289588 | −2.631225249 |
| 65 | chr13: 32034512 | −0.311623607 |
| 66 | chr13: 77838609 | 1.844834596 |
| 67 | chr13: 30455076 | −3.508163558 |
| 68 | chr13: 85584193 | −0.540711444 |
| 69 | chr13: 1535436 | −4.226227735 |
| 70 | chr13: 111038503 | −4.872094667 |
| 71 | chr14: 31839031 | −3.157679713 |
| 72 | chr14: 71122259 | −0.311791447 |
| 73 | chr16: 57712066 | −0.895052703 |
| 74 | chr17: 43961681 | −3.8209032 |
| 75 | chr18: 17893916 | −3.998631584. |

Further, on the basis of the technical solutions provided in this application, in the model: age in days=w₁·β₁+w₂·β₂+ . . . wᵢ·βᵢ+w₇₅·β₇₅+383.90, wherein wᵢ is the weight of CpG site i, βᵢ is the methylation level of site i.

Further, the methylation levels of the biomarker CpG sites are measured by measuring the methylation levels of CpG sites in the genome of a biological sample.

Further, wherein the biological sample is muscle, blood, saliva, epidermis, brain, kidney or liver sample of pigs, preferably pig muscle.

In one embodiment of the present application, the method for predicting age in days of pigs includes the following steps:
Step 1: extracting the genomic DNA of a biological sample;
Step 2: performing whole genome methylation sequencing on the extracted genomic DNA;
Step 3: calculating the methylation levels of corresponding sites in samples of different ages in days;
Step 4: constructing an Elastic Net linear regression model for predicting age in days;
Step 5: identifying CpG sites for predicting age in days;
Step 6: determining the weight of each CpG site;
Step 7: verifying the accuracy of the determined sites in the sample and the model.

The applicant obtains a method for predicting age in days of pigs based on DNA methylation levels through research. In this method, 75 CpG sites on pigs genome are screened and identified, and a corresponding weight value for each CpG site is calculated. A linear regression model for predicting age in days of pigs is constructed according to the methylation levels of these 75 CpG sites and the corresponding weights.

The above-mentioned technical solutions according to the application have the following beneficial effects:
(1) The above-mentioned biomarkers provided in this application can be used to predict the growth age in days of pigs, which provides a new idea for the mechanism study of pig meat production traits, and is beneficial to molecular design breeding of pigs.
(2) The method for predicting growth age in days of pigs based on CpG methylation according to the present application fills the gap in the age prediction model of pigs based on DNA methylation, and provides an ideal model for investigating important scientific issues such as development and aging of humans and animals.
(3) The model for predicting growth age in days of pigs based on CpG methylation provided by the present application has high accuracy, and is accurate and reliable in detecting age in days of pigs.

### BRIEF DESCRIPTION OF THE DRAWINGS

FIG. 1 is a graph showing the comparison between the predicted apparent age in days and the actual age in days based on methylation sites in the model constructed in Example 2.

### DETAILED DESCRIPTION OF THE EMBODIMENTS

Unless otherwise defined, all scientific and technical terms used in this application have the same meaning as commonly understood by an ordinary skilled person in the art of this application.

The technical solutions of the examples according to the present application will be clearly and completely described below with reference to the accompanying drawings in the examples of the present application. Obviously, the described examples are only a part of the examples of the present application, but not all of the examples. Based on the examples in the present application, all other examples obtained by the ordinary skilled person in the art without creative efforts shall fall within the protection scope of the present application.

Unless otherwise specified, the materials, reagents, etc. used in the following examples are commercially available.

The present application will be described in detail below with reference to specific examples, which are used to understand rather than limit the present application.

As used herein, the term “biomarker” refers to a CpG site that may be methylated. Methylation typically occurs in a CpG-containing nucleic acid. A CpG-containing nucleic acid may be present, for example, in a CpG island, a CpG dinucleotide, a promoter, an intron, or an exon of a gene.

As used herein, the term “DNA methylation” refers to the addition of a methyl group to the 5′-carbon of a cytosine residue between CpG dinucleotides (i.e., 5-methylcytosine). DNA methylation can occur at cytosines in other contexts, such as CHG and CHH, wherein H is adenine, cytosine, or thymine. Cytosine methylation can also be in the form of 5-hydroxymethylcytosine. DNA methylation can include non-cytosine methylation, such as N6-methyladenine.

As used herein, the term “genome” or “genomic” refers to all genetic material in the chromosomes of an organism. DNA derived from the genetic material in the chromosomes of a particular organism is genomic DNA.

As used herein, the term “gene” refers to a region of genomic DNA associated with a specified gene. For example, such a region can be defined by a specific gene (such as an exon, an intron, and a control sequence for associated expression) and its flanking sequences. However, it has been recognized in the art that methylation in a specific region is often indicative of methylation status at a proximal genomic locus.

### EXAMPLE 1

A method for constructing a model for predicting age in days of pigs, including the following steps:

### 1. Extraction of Pig Genomic DNA

The muscle tissues of the experimental pigs are sampled and lysed with 0.5 mL of lysis buffer (0.5 mol/L EDTA, 1 mol/L NaCl, 10% SDS, RNase stock), digesting with 10 μl of proteinase K (5 mg/ml), and extracting DNA by phenol imitation method. The specific steps are as follows:
(1) cutting the tissues into pieces to add to a 1.5 mL centrifuge tube, adding lysis buffer and proteinase K to the tube, then placing on a shaker (56° C., 5 h);
(2) adding an equal volume of Tris-saturated phenol (500 μL) and shaking (10 min);
(3) centrifuging at 12000 rpm for 5 min, and transferring the upper layer liquid to a new centrifuge tube;
(4) preparing a mixed solution of Tris-saturated phenol:chloroform:isoamyl alcohol=25:24:1;
(5) adding 0.45 mL of the mixed solution of above step (4) to the new centrifuge tube containing the supernatant;
(6) centrifuging at 12,000 rpm for 5 min, transferring the supernatant to a new centrifuge tube, and adding an equal volume (0.4 mL) of a mixture of chloroform and isoamylol (chloroform:isoamylol=24:1);
(7) centrifuging at 12000 rpm for 5 min, transferring the supernatant to a new centrifuge tube, adding 2.5 times of absolute ethanol pre-cooled at −20° C., and staying at −20° C. overnight;
(8) centrifuging at 12,000 rpm for 5 min, discarding the supernatant to retain the white precipitate, adding 0.4 mL of 75% ethanol, pipetting repeatedly, and centrifuging to remove the liquid;
(9) repeating step (8);
(10) adding ddH₂O to complete the extraction.

### 2. Whole-Genome Methylation Sequencing and Calculate the Methylation Levels of CpG Sites

The whole-genome methylation sequencing results are compared to calculate the methylation levels of CpG sites. The specific methods are as follows:
(1) The genomic DNA extracted in the previous step is randomly broken into 200-300 bp by using Covaris S220; the broken DNA fragments are subjected to end repair, A tail addition, and connected with sequencing linker in which all cytosines are modified by methylation.
(2) Then DNA was treated with bisulfate using EZ DNA Methylation Gold Kit, Zymo Research; after the treatment, unmethylated Cytosine (C) is converted to Uracil (U) (after PCR amplification, U becomes Thymine (T)), while methylated C remains unchanged, and then PCR amplification is performed to obtain the final DNA library.
(3) Illumina sequencing is performed on the DNA library, and the sequencing platform is HiSeq X Ten. The methylation sites are detected by Bismark, and the methylation levels of the identified methylation sites are calculated.

### 3. Construction of a Linear Model for Predicting Age in Days of Pigs. The Model is as Follows

Age in days=w₁·β₁+w₂·β₂+ . . . wᵢ·βᵢ+w₇₅·β₇₅+383.90, wherein wᵢ is the weight of CpG site i, βᵢ is the methylation level at site i.

See Table 1 for the CpG sites and weight information.

TABLE 1

| Number (i) | CpG position information (β) | Weight (w) |
|---|---|---|
| 1 | chr1: 265469121 | −0.19791914 |
| 2 | chr1: 6993958 | −3.224485644 |
| 3 | chr1: 77278255 | −13.28624592 |
| 4 | chr1: 90279146 | −9.413975275 |
| 5 | chr1: 10222822 | −2.319516222 |
| 6 | chr1: 200765194 | 6.224564956 |
| 7 | chr1: 252703561 | −10.29425473 |
| 8 | chr1: 127811329 | −0.288286911 |
| 9 | chr1: 218682018 | −8.74861671 |
| 10 | chr1: 272166208 | −0.958636654 |
| 11 | chr2: 112726051 | −0.00030695 |
| 12 | chr2: 131821312 | −1.487907119 |
| 13 | chr3: 79519033 | −1.427572944 |
| 14 | chr3: 71354421 | −14.56809668 |
| 15 | chr3: 96708114 | −5.697719601 |
| 16 | chr3: 4786944 | −6.781267851 |
| 17 | chr4: 110707399 | −0.007481015 |
| 18 | chr4: 51236025 | −1.595911641 |
| 19 | chr4: 61693637 | −1.027410147 |
| 20 | chr4: 35277986 | −0.049404384 |
| 21 | chr4: 71941843 | −13.62773853 |
| 22 | chr4: 38392750 | −0.043794313 |
| 23 | chr5: 46167692 | −2.61890723 |
| 24 | chr5: 3442060 | −14.13370338 |
| 25 | chr5: 83823568 | −1.940844913 |
| 26 | chr5: 86678792 | −8.038210429 |
| 27 | chr6: 63915584 | −6.430323147 |
| 28 | chr6: 98241827 | −19.83015838 |
| 29 | chr6: 7667231 | −0.115183771 |
| 30 | chr6: 59654560 | −0.010556261 |
| 31 | chr6: 148902979 | −13.09889713 |
| 32 | chr6: 131779338 | −0.016545453 |
| 33 | chr6: 131779339 | −2.563888441 |
| 34 | chr6: 63915581 | −7.790688318 |
| 35 | chr6: 151183086 | −2.317710899 |
| 36 | chr6: 107410789 | −7.746859508 |
| 37 | chr6: 134649996 | −42.41052359 |
| 38 | chr7: 15916877 | −5.765286814 |
| 39 | chr7: 1722548 | −1.232989258 |
| 40 | chr7: 89164845 | −1.78588923 |
| 41 | chr7: 14846023 | −1.915909405 |
| 42 | chr7: 70113867 | −5.225256985 |
| 43 | chr7: 89164756 | −0.102078131 |
| 44 | chr7: 86102364 | −1.624811107 |
| 45 | chr7: 89164755 | −4.012719139 |
| 46 | chr8: 46226086 | −3.368393933 |
| 47 | chr8: 71696260 | −17.09415973 |
| 48 | chr8: 138571452 | −19.74938423 |
| 49 | chr8: 78759323 | −5.382316805 |
| 50 | chr8: 116621205 | −4.395514047 |
| 51 | chr8: 41380820 | −0.033290161 |
| 52 | chr9: 116669694 | −0.979621002 |
| 53 | chr9: 68467395 | −1.528021515 |
| 54 | chr9: 96069192 | −9.073121614 |
| 55 | chr9: 36094595 | −15.79167462 |
| 56 | chr9: 73739560 | −1.061762087 |
| 57 | chr9: 114311129 | −0.276923385 |
| 58 | chr10: 14130890 | −0.047930706 |
| 59 | chr10: 14130912 | −0.872727299 |
| 60 | chr10: 27158773 | −8.310078727 |
| 61 | chr11: 43923343 | −5.381489916 |
| 62 | chr11: 13802486 | −2.727387937 |
| 63 | chr12: 52792396 | −6.930884723 |
| 64 | chr13: 158289588 | −2.631225249 |
| 65 | chr13: 32034512 | −0.311623607 |
| 66 | chr13: 77838609 | 1.844834596 |
| 67 | chr13: 30455076 | −3.508163558 |
| 68 | chr13: 85584193 | −0.540711444 |
| 69 | chr13: 1535436 | −4.226227735 |
| 70 | chr13: 111038503 | −4.872094667 |
| 71 | chr14: 31839031 | −3.157679713 |
| 72 | chr14: 71122259 | −0.311791447 |
| 73 | chr16: 57712066 | −0.895052703 |
| 74 | chr17: 43961681 | −3.8209032 |
| 75 | chr18: 17893916 | −3.998631584. |

### EXAMPLE 2

### Verification of the Accuracy of the CpG Sites and the Model in Example 1

### 1. Extraction of Pig Genomic DNA, Whole-Genome Methylation Sequencing

The skeletal muscle tissues of the experimental pigs at 27 time points are sampled, with 3 replicates for each time point, for a total of 81 samples, wherein 80% of the samples (n=64) are randomly selected as training samples, and the remaining 20% of the samples (n=17) as test verification samples. 0.5 mL lysis buffer (0.5 mol/EDTA, 1 mol/L NaCl, 10% SDS, RNase stock) is used for lysis, digesting with 10 μL of proteinase K (5 mg/mL), and extracting DNA by phenol imitation method. The specific steps are as follows:
(1) cutting the tissue into pieces to add to a 1.5 mL centrifuge tube, adding lysis buffer and proteinase K to the tube, then placing on a shaker (56° C., 5 h);
(2) adding an equal volume of Tris-saturated phenol (500 μL) and shaking (10 min);
(3) centrifuging at 12000 rpm for 5 min, and transferring the upper layer liquid to a new centrifuge tube;
(4) preparing a mixed solution of Tris-saturated phenol:chloroform:isoamyl alcohol=25:24:1;
(5) adding 0.45 mL of the mixed solution of above step (4) to the new centrifuge tube containing the supernatant;
(6) centrifuging at 12,000 rpm for 5 min, transferring the supernatant to a new centrifuge tube, and adding an equal volume (0.4 mL) of a mixture of chloroform and isoamylol (chloroform:isoamylol=24:1);
(7) centrifuging at 12000 rpm for 5 min, transferring the supernatant to a new centrifuge tube, adding 2.5 times of absolute ethanol pre-cooled at −20° C., and staying at −20° C. overnight;
(8) centrifuging at 12,000 rpm for 5 min, discarding the supernatant to retain the white precipitate, adding 0.4 mL of 75% ethanol, pipetting repeatedly, and centrifuging to remove the liquid;
(9) repeating step (8);
(10) adding ddH₂O to complete the genomic DNA extraction.

### 2. Whole-Genome Methylation Sequencing and Calculate the Methylation Levels of CpG Sites

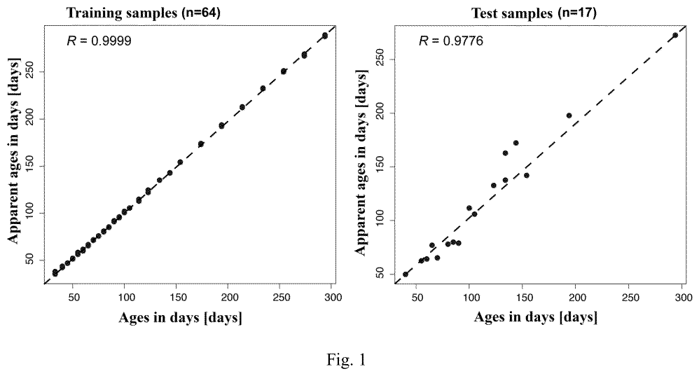

(1) The genomic DNA is randomly broken into 200-300 bp by using Covaris S220; the broken DNA fragments are subjected to end repair, A tail addition, and connected with sequencing linker in which all cytosines are modified by methylation.
(2) Then DNA was treated with bisulfite using EZ DNA Methylation Gold Kit, Zymo Research; after the treatment, unmethylated Cytosine (C) is converted to Uracil (U) (after PCR amplification, U becomes Thymine (T)), while methylated C remains unchanged, and then PCR amplification is performed to obtain the final DNA library.
(3) Illumina sequencing is performed on the DNA library, and the sequencing platform is HiSeq X Ten. The methylation sites are detected by Bismark, and the methylation levels of the identified methylation sites are calculated.
(4) The methylation level data of randomly selected 64 samples with different ages in days is used as test data to construct a model, and the data of the remaining 17 samples with different ages in days is used as verification data; the speculated ages in days are calculated according to the constructed model, comparing them with the actual ages in days (the comparison results are shown in FIG. 1) to test the accuracy of the model. The results in FIG. 1 show that, in the training population, the median absolute error of the apparent and actual ages in days for 61 samples is 1.22 days, and the correlation of age in days is 0.9999. In the test population, the median absolute errors of apparent and actual ages in days before and after birth for 21 samples are respectively 6.3 and 12.06 days, and the correlation of age in days is 0.9776. It is proved that the constructed model has high accuracy, and the methylation information of the selected 75 CpG sites can be used to effectively predict age in days of pigs.

The above descriptions are only preferred examples of the present application, and are not intended to limit the present application. Any modifications, equivalent replacements, etc. made within the spirit and principles of the present application shall be encompassed in the protection scope of the present application.

What is claimed is:

1. A method for predicting age in days of a pig, comprising measuring the methylation levels of 75 CpG sites in genomic DNA of a pig, and utilizing a statistical prediction algorithm to determine age in days of a pig; the statistical prediction algorithm comprises: (a) obtaining a linear combination of methylation levels 75 CpG sites, the method for obtaining the linear combination comprises: the methylation levels of the CpG sites and the corresponding weights of each CpG site are used to construct an Elastic Net linear regression model; and (b) applying a transformation to the linear combination to determine age in days of a pig
wherein, position informations of the 75 CpG sites as following: chr1:265469121, chr1:6993958, chr1:77278255, chr1:77278255, chr1:90279146, chr1:10222822, chr1:200765194, chr1:252703561, chr1:127811329, chr1:218682018, chr1:272166208, chr2:112726051, chr2:131821312, chr3:79519033, chr3:71354421, chr3:96708114, chr3:4786944, chr4:110707399, chr4:51236025, chr4:61693637, chr4:35277986, chr4:71941843, chr4:38392750, chr5:46167692, chr5:3442060, chr5:83823568, chr5:86678792, chr6:63915584, chr6:98241827, chr6:7667231, chr6:59654560, chr6:148902979, chr6:131779338, chr6:131779339, chr6:63915581, chr6:151183086, chr6:107410789, chr6:134649996, chr7:15916877, chr7:1722548, chr7:89164845, chr7:14846023, chr7:70113867, chr7:89164756, chr7:86102364, chr7:89164755, chr8:46226086, chr8:71696260, chr8:138571452, chr8:78759323, chr8:116621205, chr8:41380820, chr9:116669694, chr9:68467395, chr9:96069192, chr9:36094595, chr9:73739560, chr9:114311129, chr10:14130890, chr10:14130912, chr10:27158773, chr11:43923343, chr11:13802486, chr12:52792396, chr13:158289588, chr13:32034512, chr13:77838609, chr13:30455076, chr13:85584193, chr13:1535436, chr13:111038503, chr14:31839031, chr14:71122259, chr16:57712066, chr17:43961681, chr18:17893916;
and, weight informations of the 75 CpG sites as following:

| Number (i) | CpG position information (β) | Weight (w) |
|---|---|---|
| 1 | chr1: 265469121 | −0.19791914 |
| 2 | chr1: 6993958 | −3.224485644 |
| 3 | chr1: 77278255 | −13.28624592 |
| 4 | chr1: 90279146 | −9.413975275 |
| 5 | chr1: 10222822 | −2.319516222 |
| 6 | chr1: 200765194 | 6.224564956 |
| 7 | chr1: 252703561 | −10.29425473 |
| 8 | chr1: 127811329 | −0.288286911 |
| 9 | chr1: 218682018 | −8.74861671 |
| 10 | chr1: 272166208 | −0.958636654 |
| 11 | chr2: 112726051 | −0.00030695 |
| 12 | chr2: 131821312 | −1.487907119 |
| 13 | chr3: 79519033 | −1.427572944 |
| 14 | chr3: 71354421 | −14.56809668 |
| 15 | chr3: 96708114 | −5.697719601 |
| 16 | chr3: 4786944 | −6.781267851 |
| 17 | chr4: 110707399 | −0.007481015 |
| 18 | chr4: 51236025 | −1.595911641 |
| 19 | chr4: 61693637 | −1.027410147 |
| 20 | chr4: 35277986 | −0.049404384 |
| 21 | chr4: 71941843 | −13.62773853 |
| 22 | chr4: 38392750 | −0.043794313 |
| 23 | chr5: 46167692 | −2.61890723 |
| 24 | chr5: 3442060 | −14.13370338 |
| 25 | chr5: 83823568 | −1.940844913 |
| 26 | chr5: 86678792 | −8.038210429 |
| 27 | chr6: 63915584 | −6.430323147 |
| 28 | chr6: 98241827 | −19.83015838 |
| 29 | chr6: 7667231 | −0.115183771 |
| 30 | chr6: 59654560 | −0.010556261 |
| 31 | chr6: 148902979 | −13.09889713 |
| 32 | chr6: 131779338 | −0.016545453 |
| 33 | chr6: 131779339 | −2.563888441 |
| 34 | chr6: 63915581 | −7.790688318 |
| 35 | chr6: 151183086 | −2.317710899 |
| 36 | chr6: 107410789 | −7.746859508 |
| 37 | chr6: 134649996 | −42.41052359 |
| 38 | chr7: 15916877 | −5.765286814 |
| 39 | chr7: 1722548 | −1.232989258 |
| 40 | chr7: 89164845 | −1.78588923 |
| 41 | chr7: 14846023 | −1.915909405 |
| 42 | chr7: 70113867 | −5.225256985 |
| 43 | chr7: 89164756 | −0.102078131 |
| 44 | chr7: 86102364 | −1.624811107 |
| 45 | chr7: 89164755 | −4.012719139 |
| 46 | chr8: 46226086 | −3.368393933 |
| 47 | chr8: 71696260 | −17.09415973 |
| 48 | chr8: 138571452 | −19.74938423 |
| 49 | chr8: 78759323 | −5.382316805 |
| 50 | chr8: 116621205 | −4.395514047 |
| 51 | chr8: 41380820 | −0.033290161 |
| 52 | chr9: 116669694 | −0.979621002 |
| 53 | chr9: 68467395 | −1.528021515 |
| 54 | chr9: 96069192 | −9.073121614 |
| 55 | chr9: 36094595 | −15.79167462 |
| 56 | chr9: 73739560 | −1.061762087 |
| 57 | chr9: 114311129 | −0.276923385 |
| 58 | chr10: 14130890 | −0.047930706 |
| 59 | chr10: 14130912 | −0.872727299 |
| 60 | chr10: 27158773 | −8.310078727 |
| 61 | chr11: 43923343 | −5.381489916 |
| 62 | chr11: 13802486 | −2.727387937 |
| 63 | chr12: 52792396 | −6.930884723 |
| 64 | chr13: 158289588 | −2.631225249 |
| 65 | chr13: 32034512 | −0.311623607 |
| 66 | chr13: 77838609 | 1.844834596 |
| 67 | chr13: 30455076 | −3.508163558 |
| 68 | chr13: 85584193 | −0.540711444 |
| 69 | chr13: 1535436 | −4.226227735 |
| 70 | chr13: 111038503 | −4.872094667 |
| 71 | chr14: 31839031 | −3.157679713 |
| 72 | chr14: 71122259 | −0.311791447 |
| 73 | chr16: 57712066 | −0.895052703 |
| 74 | chr17: 43961681 | −3.8209032 |
| 75 | chr18: 17893916 | −3.998631584 |

and, the Elastic Net linear regression model is: age in days=w₁·β₁+w₂·β₂+ . . . wᵢ·βᵢ+w₇₅·β₇₅+383.90, wherein wᵢ is the weight information of CpG site i, βᵢ is the methylation level of site i.

2. The method according to claim 1, wherein the version of the pig reference genome used in the model is Sscrofa11.1 version.

3. The method according to claim 1, wherein the methylation levels of the biomarker CpG sites are measured by measuring the methylation levels of CpG sites in the genome of the biological sample, wherein the biological sample is a muscle, blood, saliva, epidermis, brain, kidney or liver sample of a pig.
